(19) United States
(12) Patent Application Publication
(10) Pub. No.: US 2024/0225658 A1
(43) Pub. Date: Jul. 11, 2024

(54) TREATMENT METHOD AND TREATMENT SYSTEM

(71) Applicant: OLYMPUS MEDICAL SYSTEMS CORP., Tokyo (JP)

(72) Inventors: Kotaro NAKAMURA, Isehara-shi (JP); Masato NARISAWA, Hino-shi (JP); Ryosuke ITO, Kokubunji-shi (JP)

(21) Appl. No.: 18/404,347

(22) Filed: Jan. 4, 2024

Related U.S. Application Data
(60) Provisional application No. 63/478,815, filed on Jan. 6, 2023.

(51) Int. Cl. A61B 17/12; A61B 17/00

(52) U.S. Cl. CPC A61B 17/12136; A61B 2017/00557; A61B 2017/00818; A61B 2017/00982; A61B 17/12099; A61B 2217/005

(57) ABSTRACT

A treatment method according to one aspect of the present disclosure may include inserting a first closing member and a second closing member into a tubular organ having at least one concaved area, and closing a part of the tubular organ with the first closing member and the second closing member in a state where the at least one concaved area is located between the first closing member and the second closing member. The method may further include disposing a holding member between the first closing member and the second closing member, and suctioning gas within the part of the tubular organ between the first closing member and the second closing member and contacting the holding member with a portion of the at least one concaved area.

### CROSS-REFERENCE TO RELATED APPLICATIONS

The present application claims priority based on U.S. Patent Provisional Application No. 63/478,815 provisionally filed in the United States on Jan. 6, 2023, the contents of which are incorporated herein by reference.

### TECHNICAL FIELD

The present invention relates to a treatment method and a treatment system.

### BACKGROUND

Colorectal diverticula are, in principle, a benign disease, but may bleed due to inflammation or local stress. If bleeding cannot be stopped with conservative treatments such as fasting and resting the intestines, endoscopic hemostatic treatment (such as clip hemostasis) is required. When bleeding occurs in multiple colonic diverticula, it is difficult to identify the bleeding point by endoscopic observation, for example, and hemostatic treatment becomes complicated.

There is a technique for administering drugs to a certain area within a tubular organ. Japanese Unexamined Patent Application, First Publication No. H5-42224 (Patent Document 1) discusses a device including two balloons. Two balloons are expanded within a blood vessel, and a drug such as heparin can be administered through an opening provided between the balloons into a space where blood flow is blocked.

### SUMMARY

When administering a drug to a certain area within a tubular organ in order to stop bleeding in a colonic diverticulum, it is necessary to administer a large amount of the drug to a certain area within the tubular organ. If the drug does not reach the inside of the colonic diverticulum, it will not be possible to sufficiently stop the bleeding in the colonic diverticulum.

Based on the above circumstances, the present disclosure provides a treatment method and a treatment device that can more easily stop bleeding from colonic diverticula.

A treatment method according to a first aspect of the present disclosure may include: inserting a first closing member and a second closing member into a tubular organ having at least one concaved area, and closing a part of the tubular organ with the first closing member and the second closing member in a state where the at least one concaved area is located between the first closing member and the second closing member. The method may further include disposing a holding member between the first closing member and the second closing member, and suctioning gas within the part of the tubular organ between the first closing member and the second closing member and contacting the holding member with a portion of the at least one concaved area.

A treatment system according to a second aspect of the present disclosure may include a first closing member and a second closing member capable of closing a part of a tubular organ. The system may further include a tube having a suction lumen, a suction pump connected to the suction lumen, and a holding member disposed between the first closing member and the second closing member. A first end of the holding member may be configured to contact with one of the first closing member and the second closing member The first closing member and the second closing member may be attached to the tube.

Thus, a potential advantage of the present treatment method and treatment system is that colonic diverticulum bleeding may be stopped more easily when administering a drug within a tubular organ in order to stop bleeding in a colonic diverticulum.

### BRIEF DESCRIPTION OF DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a schematic diagram of a treatment system used in a treatment method according to a first embodiment.

FIG. 2 illustrates an example of a cross-sectional view of a treatment device taken along line I-I in FIG. 1.

FIG. 3 illustrates an example of a cross-sectional view of the treatment device taken along line II-II in FIG. 1.

FIG. 4 illustrates an example of a schematic diagram showing step A of the treatment method.

FIG. 5 illustrates an example of a schematic diagram showing step B of the treatment method.

FIG. 6 illustrates an example of a schematic diagram showing step C and step D of the treatment method.

FIG. 7 illustrates an example of a schematic diagram showing step C using a treatment device that does not include a holding member.

FIG. 8 illustrates an example of a schematic diagram of a modification of the treatment device.

FIG. 9 illustrates an example of a schematic diagram of another modification of the treatment device.

FIG. 10 illustrates an example of a schematic diagram of another modification of the treatment device.

FIG. 11 illustrates an example of a schematic diagram of the same modification of the treatment device.

FIG. 12 illustrates an example of a schematic diagram of another modification of the treatment device.

FIG. 13 illustrates an example of a schematic diagram of another modification of the treatment device.

### DETAILED DESCRIPTION

### First Embodiment

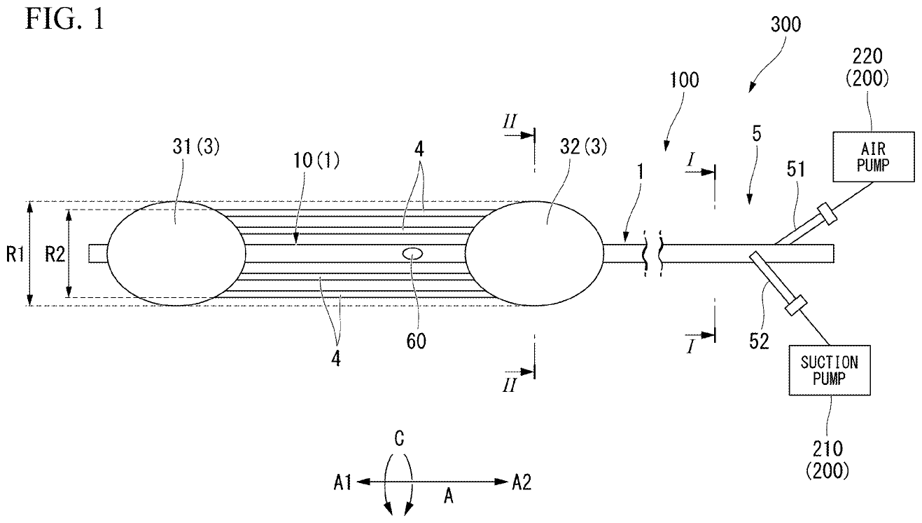

FIG. 1 illustrates an example of schematic diagram of a treatment system 300 used in a treatment method according to this embodiment. Although the treatment method according to this embodiment is not limited to using the treatment system 300, it can be suitably performed by using the treatment system 300.

The treatment system 300 is a system that can treat an affected area within a tubular organ. The treatment system 300 may include a treatment device 100, a suction pump 210, and an air pump 220. The suction pump 210 and the air pump 220 may be an integrated treatment device control system 200.

The treatment device 100 may include a long tube 1, a closing member 3, a holding member 4, and an operating section 5. In the following description, in the longitudinal direction A of the treatment device 100, the side inserted into the patient's body is referred to as the “tip side (distal side) A1”, and the side of the operating section 5 is referred to as the “base end side (proximal side) A2”.

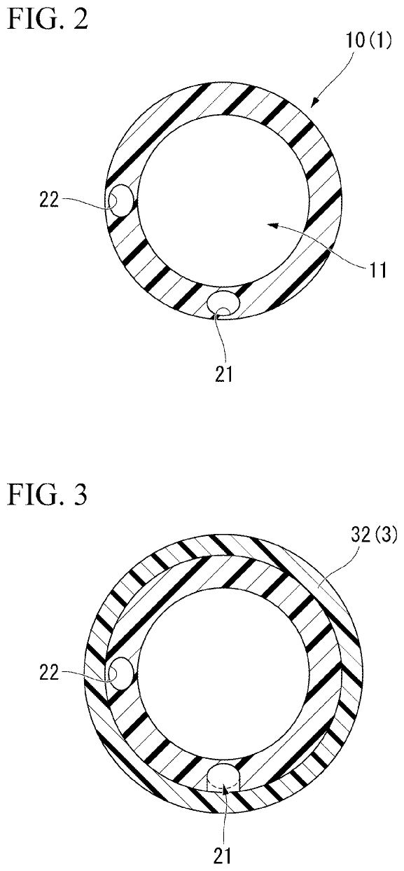

FIG. 2 illustrates an example of a cross-sectional view of the treatment device 100 taken along line I-I in FIG. 1.

As shown in FIG. 2, the tube 1 may be a multi-lumen tube having, for example, three lumens: a main lumen 11 with the largest inner diameter and two sub-lumens (first lumen 21 and second lumen 22) formed within the wall. The tube 1 may be connected to the operating section 5 at the proximal side A2. The tube 1 may include a suction lumen 22.

The closing member 3 is a member that may close a certain range within the tubular organ. This embodiment may include a first balloon 31 and a second balloon 32 provided closer to the proximal side A2 than the first balloon 31. The first closing member 31 and the second closing member 32 is configured to close a part of a tubular organ, and the first closing member 31 and the second closing member 32 attached to the tube 1. The first closing member 31 is provided distally relative to the second closing member 32. The holding member 4 may have a first length in a longitudinal direction of the tube 1, and a portion of the tube 1 may have a second length between a proximal end of the first closing member 31 and a distal end of the second closing member 32. The first length can be longer than the second length when the holding member 4 is contact with the object. The first length can be longer than the second length when the holding member 4 is expanded. The first length can be longer than the second length when the gas is suctioned.

The first balloon (first closing member) 31 may be attached to the outer peripheral surface of a distal end portion 10 of the tube 1. The first balloon 31 may be provided closer to the distal side A1 than the second balloon 32. The first balloon 31 can be made of an easily extensible material such as silicone or elastomer.

The second balloon (second closing member) 32 may be attached to the outer peripheral surface of the distal end portion 10 of the tube 1. The second balloon 32 may be provided closer to the proximal side A2 than the first balloon 31. The second balloon 32 may be made of an easily extensible material such as silicone or elastomer.

FIG. 3 illustrates an example of a cross-sectional view of the treatment device 100 taken along line II-II in FIG. 1.

The first lumen (inflation lumen) 21 may open on the outer peripheral surface of the tube 1 at a portion where the first balloon 31 and the second balloon 32 are attached and may communicate with (e.g., touch, contact, be attached to, be connected to or with, or the like) the first balloon 31 and the second balloon 32. Although FIG. 3 shows a portion where the first lumen 21 and the second balloon 32 are in communication, the first lumen 21 and the first balloon 31 are also in communication in a similar manner. The first lumen 21 may extend to the first port 51 of the operating section 5. The air pump 220 may be connected to the first port 51. The air pump 220 can inflate the first balloon 31 and the second balloon 32 by supplying fluid to the first port 51. The first balloon 31 and the second balloon 32 may communicate with different lumens. In this case, the first balloon 31 and the second balloon 32 can be expanded and deflated independently.

The second lumen (suction lumen) 22 may communicate with an opening (suction port, suction opening) 60 provided on the outer peripheral surface of the tube 1 between the first balloon 31 and the second balloon 32. The second lumen 22 may extend to a second port 52 provided in the operating section 5. The suction pump 210 may be connected to the second port 52. The suction pump 210 can suction fluid from the opening 60 by suctioning the fluid into the second port 52. The tube 1 includes the suction opening 60 disposed between the first closing member 31 and the second closing member 32.

The holding member (lumen maintenance structure) 4 is a member that may be provided between the first balloon 31 and the second balloon 32. In this embodiment, the holding member 4 may be a plurality of linear members, the distal end of which may be connected to the first balloon 31, and the proximal end of which may be connected to the second balloon 32. The holding member 4 may be disposed along the circumferential direction C around the distal end portion 10 of the tube 1. The holding member 4 can be disposed between the first closing member 31 and the second closing member 32, and a first end of the holding member 4 can be configured to contact with one of the first closing member 31 and the second closing member 32. The holding member 4 is expandable together with the first closing member 31 and the second closing member 32. The first end of the holding member 4 can be configured to be connected to the first closing member 31 and a second end of the holding member 4 is connected to the second closing member 32.

The holding member 4 is a member that may be formed linearly along the longitudinal direction A. The holding member 4 may be, for example, an elastic member such as a metal wire or rubber.

The holding member 4 may expand (increases in diameter) in the radial direction as the first balloon 31 and the second balloon 32 expand. In other words, as the first balloon 31 and the second balloon 32 expand, the distance between the longitudinal axis of the tube 1 and the longitudinal axis of each linear member can increase. The maximum length R2 in the radial direction when the holding member 4 is expanded may be less than the maximum length R1 in the radial direction when the first balloon 31 and the second balloon 32 are expanded to the maximum (R2≤R1). The disposing of the holding member 4 includes expanding the holding member 4 together with the closing of the part of the tubular organ by the first closing member 31 and the second closing member 32.

### [Operation of Treatment System 300]

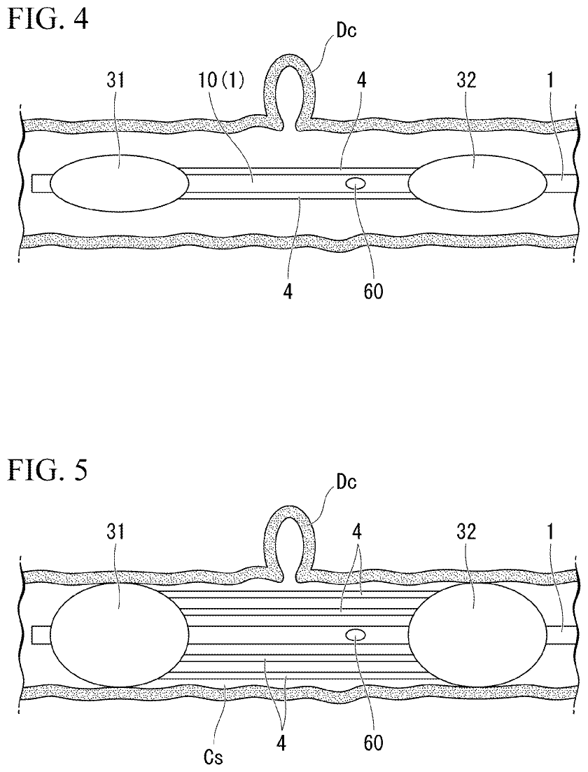

Next, with reference to FIGS. 4 to 7, the treatment method of this embodiment using the treatment system 300 will be described using an example of bleeding from a plurality of diverticula that have occurred in the large intestine. A diverticulum is a concave diseased area formed in the lumen of the large intestine.

The operator can pass the endoscope through the main lumen 11 of the tube 1. The operator can insert the endoscope into the large intestine to be treated and advance the tip of the endoscope to the area to be treated.

FIG. 4 illustrates an example of a schematic diagram showing step A of the treatment method.

Next, the operator inserts the tube 1 into the large intestine (Step A). Furthermore, the tube 1 can be advanced along the endoscope, and the distal end portion 10 of the tube 1 can reach the site to be treated. The advancement of the endoscope and the advancement of the tube 1 may be performed in parallel. The operator inserts a first closing member 31 and a second closing member 32 into the tubular organ having at least one concaved area. The concaved area may include at least one of the diverticulum Dc, the peripheral of the diverticulum Dc, opening the diverticulum Dc, surface of diverticulum Dc, vicinity of the diverticulum Dc including not diseased area, vicinity of the opening of diverticulum Dc including not diseased area. At least one concaved area includes a plurality of concaved areas.

In step A, the operator positions the distal end portion 10 of the tube 1 so that the diverticulum Dc to be treated can be located between the first balloon 31 and the second balloon 32.

FIG. 5 illustrates an example of a schematic diagram showing step B of the treatment method.

Next, the operator may operate the air pump 220 to supply gas or liquid to the first balloon 31 and the second balloon 32 to expand them (Step B). The expanded first balloon 31 and second balloon 32 contact the inner wall of the large intestine and close the lumen of the large intestine, with the diverticulum Dc to be treated located between the first balloon 31 and the second balloon 32. As a result, a closed space Cs can be formed between the first balloon 31 and the second balloon 32. The first closing member 31 and the second closing member 32 may close a part of the tubular organ in a state where the at least one concaved area is located between the first closing member 31 and the second closing member 32. The holding member 4 may be disposed between the first closing member 31 and the second closing member 32.

The holding member 4 may expand (e.g., the diameter enlarges) together with the first balloon 31 and the second balloon 32. The maximum length R2 in the radial direction when the holding member 4 can be expanded may be less than the maximum length R1 in the radial direction when the first balloon 31 and the second balloon 32 can be expanded to the maximum (R2≤R1). Therefore, the closed space Cs can be reliably formed by the two closing members (first balloon 31 and second balloon 32). When the part of the tubular organ is closed, the first closing member 31 can expand at a first length R1 in the radial direction of the tube, and/or the second closing member 32 can expand at a second length R1 in the radial direction of the tube. When the holding member 4 is disposed, the holding member 4 can be expand at a third length R2 in the radial direction of the tube. The third length R2 of the holding member 4 is equal to or less than the first radial diameter R1 of the first closing member 31 and/or the second radial diameter R1 of the second closing member 32.

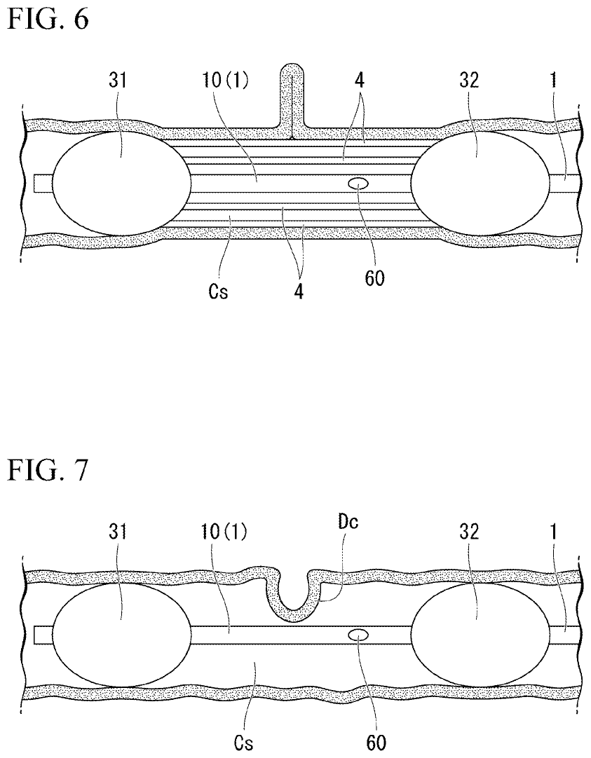

FIG. 6 illustrates an example of a schematic diagram showing Steps C and D of the treatment method.

Next, the operator may operate the suction pump 210 to suction the gas in the closed space Cs from the opening 60 (Step C). Through step C, the internal pressure of the closed space Cs decreases and becomes negative pressure. Gas within the part of the tubular organ between the first closing member 31 and the second closing member 32 may be suctioned.

The operator can continue to suction the gas in the closed space Cs and can bring the holding member 4 into contact with a portion of the inner wall of the contracted large intestine, as shown in FIG. 6 (Step D). Although the holding member 4 may come into contact with a part of the entrance of the diverticulum Dc, it does not completely block the entrance of the diverticulum Dc. As a result, the gas within the diverticulum Dc can also be sucked, and the mucous membranes within the diverticulum Dc can come into close contact with each other, thereby stopping bleeding from the diverticulum. The holding member 4 may contact a portion of the at least one concaved area. The at least one concaved area is bleeding, and the suctioning of the gas within the part of the tubular organ and the contacting of the holding member 4 stop bleeding at the at least one concaved area. The at least one concaved area located between the first closing member 31 and the second closing member 32.

FIG. 7 illustrates an example of a schematic diagram showing step C using a treatment device that does not include the holding member 4.

The treatment device shown in FIG. 7 does not have the holding member 4. Therefore, when the closed space Cs is made to have a negative pressure in step C, the diverticulum Dc that has bulged outside the lumen can be reversed and swells inside the lumen, as shown in FIG. 7. In this case, the diverticulum may not be stopped and bleeding may occur. On the other hand, in the treatment device 100, the holding member 4 can suitably prevent the diverticulum Dc from inverting and swelling inside the lumen.

According to the treatment method and treatment system 300 according to the present embodiment, diverticular bleeding can be suitably stopped without administering a large amount of drug into the tubular organ. Furthermore, even if multiple diverticula are bleeding, the bleeding can be stopped at once.

Although the first embodiment of the present invention has been described above in detail with reference to the drawings, the specific configuration is not limited to this embodiment, and design changes may be made within the scope of present disclosure. Moreover, the components shown in the above-described embodiments and the modifications shown below can be configured by appropriately combining them.

### (Modification 1)

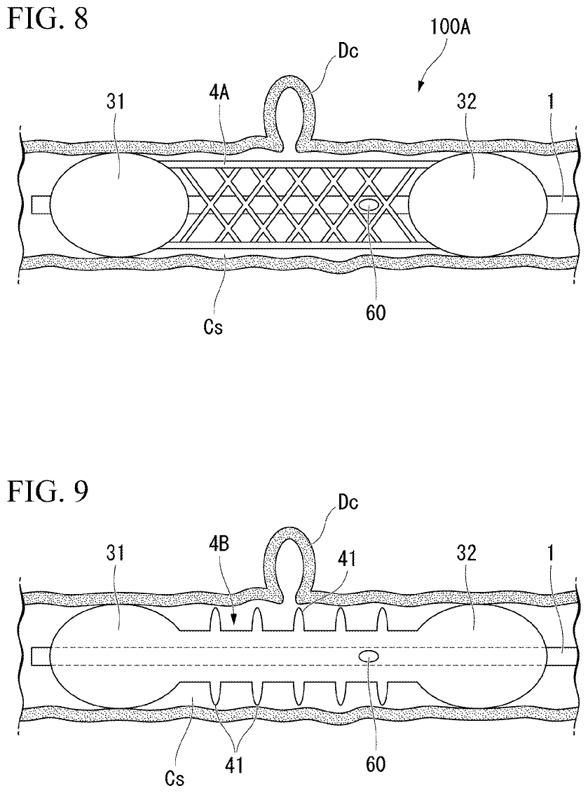

FIG. 8 illustrates an example of a schematic diagram of a treatment device 100A that is a modification of the treatment device 100. The treatment device 100A may include a long tube 1, a closing member 3, a holding member 4A, and an operating section 5. The holding member 4A may be formed in a mesh shape. The holding member 4A may be or may include, for example, a net or a stent. The holding member 4A may have a stronger lumen holding force than the holding member 4, and can reduce the burden on the large intestine in step D.

### (Modification 2)

FIG. 9 illustrates an example of a schematic diagram of a treatment device 100B that is a modification of the treatment device 100. The treatment device 100B may include a long tube 1, a closing member 3, a holding member 4B, and an operating section 5. The holding member 4B may be a balloon having a plurality of protrusions 41. The protrusion 41 can protrude in the radial direction. The holding member 4B may make contact with, connect with, attach to, touch, or otherwise communicate with at least one of the first balloon 31 and the second balloon 32. The holding member 4B may expand (increases in diameter) in the radial direction as the first balloon 31 and the second balloon 32 expand.

### (Modification 3)

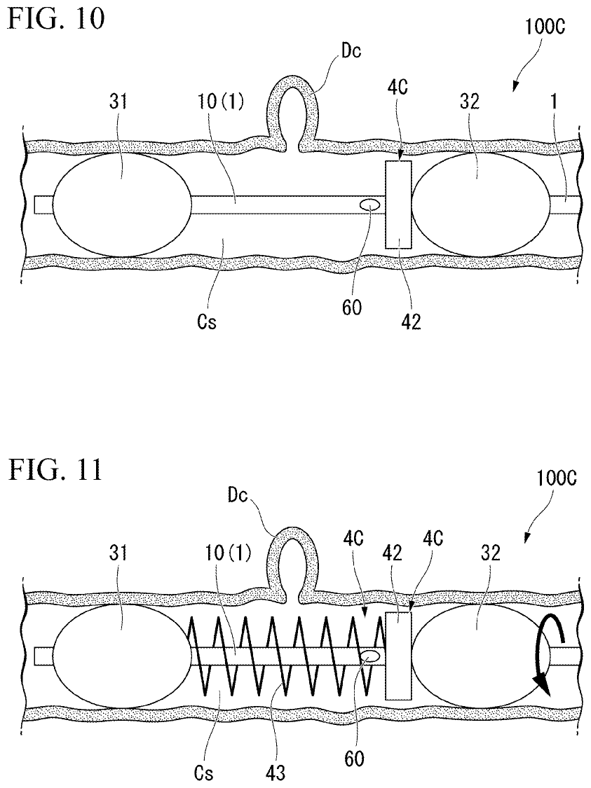

FIGS. 10 and 11 illustrate example schematic diagrams of a treatment device 100C that is a modification of the treatment device 100. The treatment device 100C may include a long tube 1, a closing member 3, a holding member 4C, and an operating section 5. The holding member 4C may include a wire accommodating portion 42 and a twisted wire 43. The wire accommodating portion 42 may be provided between the first balloon 31 and the second balloon 32. The twisted wire 43 may be stored in the wire accommodating portion 42. By rotating the knob provided on the operating section 5, the twisted wire 43, which has a tendency to twist, may pop out or extend out from the wire accommodating portion 42 and spread out in a spiral shape along the longitudinal direction A. The twisted wire 43 may be made of metal, such as a shape memory alloy such as nickel-titanium (Ni—Ti or nitinol). The helically expanded twisted wire 43 can function as a lumen maintenance structure.

### (Modification 4)

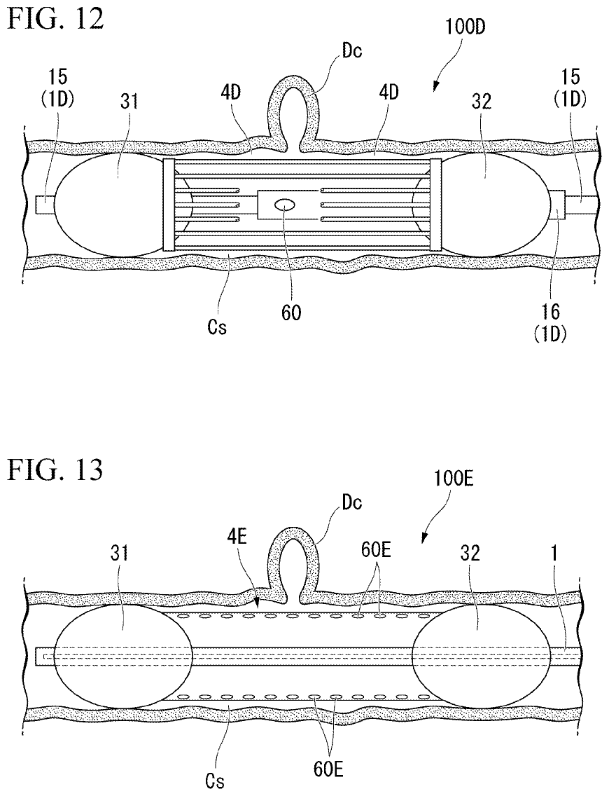

FIG. 12 illustrates an example of a schematic diagram of a treatment device 100D that is a modification of the treatment device 100. The treatment device 100D may include a long tube 1D, a closing member 3, a holding member 4D, and an operating section 5. The tube 1D may include a first tube 15 and a second tube 16 through which the first tube 15 may be inserted. The first tube 15 and the second tube 16 may be movable relative to each other in the longitudinal direction A. The first balloon 31 may be attached to the first tube 15. The second balloon 32 may be attached to the second tube 16. By moving the first tube 15 forward and backward with respect to the second tube 16, the first balloon 31 can be moved forward and backward with respect to the second balloon 32. The first closing member 31 and the second closing member 32 are movable relative to each other along the longitudinal direction of the tube 1.

The holding member 4D may be a linear member such as a wire. By advancing the first balloon 31 with respect to the second balloon 32, the holding member 4D can be maintained in a linear shape and tension can be generated. By generating tension in the holding member 4D, the lumen holding force of the holding member 4D may be strengthened. For example, after step A, the surgeon can advance the first balloon 31 relative to the second balloon 32.

### (Modification 5)

FIG. 13 illustrates an example of a schematic diagram of a treatment device 100E that is a modification of the treatment device 100. The treatment device 100E includes a long tube 1, a closing member 3, a holding member 4E, and an operating section 5. The holding member 4E is formed into a cylindrical shape and expands (increases in diameter) in the radial direction as the first balloon 31 and the second balloon 32 expand. The holding member 4E has a plurality of suction ports 60E. The treatment device 100E can suitably suck the gas in the closed space Cs using the plurality of suction ports 60E.

### Additional Notes and Examples

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

Example 1. A treatment method comprising:
a step of inserting a first closing member and a second closing member into a tubular organ having at least one concave affected area;
a step of closing the tubular organ with the first closing member and the second closing member in a state where the affected area is located between the first closing member and the second closing member;
a step of suctioning gas within the tubular organ between the first closing member and the second closing member; and
a step of contacting a holding member, which is disposed between the first closing member and the second closing member, with a portion of the affected area.

Example 2. The treatment method according to Example 1, wherein
the affected area is bleeding, and
the step of suctioning and the step of contacting stop bleeding at the affected area.

Example 3. The treatment method according to Example 2, wherein the affected area is a diverticulum.

Example 4. The treatment method according to Example 3, wherein the step of suctioning and the step of contacting stop bleeding from the diverticulum by bringing mucous membranes in the diverticulum into close contact with each other.

Example 5. The treatment method according to Example 1, wherein a plurality of the affected areas are arranged between the first closing member and the second closing member.

Example 6. The treatment method according to Example 1, wherein the step of closing expands the holding member together with the first closing member and the second closing member.

Example 7. The treatment method according to Example 1, wherein the step of closing expands the holding member such that a maximum radial length of the holding member is equal to or less than a maximum radial length of the first closing member and the second closing member.

Example 8. A treatment system comprising:
a first closing member and a second closing member capable of closing a tubular organ;
a tube having a suction lumen and to which the first closing member and the second closing member are attached;
a suction pump connected to the suction lumen; and
an expandable holding member disposed between the first closing member and the second closing member, one end connected to the first closing member and the other end connected to the second closing member.

Example 9. The treatment system according to Example 8, wherein the holding member is expandable together with the first closing member and the second closing member.

Example 10. The treatment system according to Example 8, wherein a maximum length in a radial direction when the holding member is expanded is not more than a maximum length in the radial direction when the first closing member and the second closing member are expanded to a maximum.

Example 11. The treatment system according to Example 8, wherein the holding member is a linear member.

Example 12. The treatment system according to Example 8, wherein the holding member is formed in a mesh shape.

Example 13. The treatment system according to Example 8, wherein the holding member is a balloon.

Example 14. The treatment system according to Example 8, wherein the holding member is a twisted wire.

Example 15. The treatment system according to Example 8, wherein the first closing member and the second closing member are movable relative to each other along a longitudinal direction.

What is claimed is:

1. A treatment method comprising:
inserting a first closing member and a second closing member into a tubular organ having at least one concaved area;
closing a part of the tubular organ with the first closing member and the second closing member in a state where the at least one concaved area is located between the first closing member and the second closing member;
disposing a holding member between the first closing member and the second closing member;
suctioning gas within the part of the tubular organ between the first closing member and the second closing member; and
contacting the holding member with a portion of the at least one concaved area.

2. The treatment method according to claim 1, wherein the at least one concaved area is bleeding, and wherein the suctioning of the gas within the part of the tubular organ and the contacting of the holding member stop bleeding at the at least one concaved area.

3. The treatment method according to claim 2, wherein the at least one concaved area is a diverticulum.

4. The treatment method according to claim 3, further comprising:
subsequent to the suctioning of the gas within the part of the tubular organ and the contacting of the holding member, bringing a first mucous membrane in the diverticulum into close contact with a second mucous membrane in the diverticulum.

5. The treatment method according to claim 1, wherein the at least one concaved area includes a plurality of concaved areas located between the first closing member and the second closing member.

6. The treatment method according to claim 1, wherein the disposing of the holding member includes expanding the holding member together with the closing of the part of the tubular organ by the first closing member and the second closing member.

7. The treatment method according to claim 1, wherein the closing of the part of the tubular organ includes expanding the first closing member at a first length and the second closing member at a second length in a radial direction of the tube,
the disposing of the holding member includes expanding the holding member at a third length in the radial direction of the tube, and
the third length of the holding member is equal to or less than the first radial diameter of the first closing member and the second radial diameter of the second closing member.

8. A treatment system comprising:
a tube including a suction lumen; and
a first closing member and a second closing member configured to close a part of a tubular organ, and the first closing member and the second closing member attached to the tube; and
a holding member disposed between the first closing member and the second closing member, wherein a first end of the holding member is configured to contact with one of the first closing member and the second closing member.

9. The treatment system according to claim 8, wherein the holding member is expandable together with the first closing member and the second closing member.

10. The treatment system according to claim 8, wherein a maximum length in a radial direction of the tube when the holding member is expanded is not more than a maximum length in the radial direction when the first closing member and the second closing member are expanded to a maximum.

11. The treatment system according to claim 8, wherein the holding member is configured to be at least one of a linear member, a net, a stent, a balloon, and a twisted wire.

12. The treatment system according to claim 8, wherein the holding member is formed in a mesh shape.

13. The treatment system according to claim 8, wherein the tube includes a suction opening disposed between the first closing member and the second closing member.

14. The treatment system according to claim 8, wherein the first end of the holding member is configured to be connected to the first closing member and a second end of the holding member is connected to the second closing member.

15. The treatment system according to claim 8, wherein the first closing member and the second closing member are movable relative to each other along a longitudinal direction of the tube.

16. The treatment system according to claim 8, wherein the first closing member is expandable at a first length in a radial direction of the tube,
the second closing member is expandable at a second length in the radial direction,
the holding member is expandable at a third length in the radial direction, and
the third length of the holding member is equal to or less than the first radial diameter of the first closing member and the second radial diameter of the second closing member.

17. The treatment system according to claim 16, the holding member is expandable together with the first closing member and the second closing member,

18. The treatment system of claim 16, wherein the first closing member is provided distally relative to the second closing member,
the holding member has a first length in a longitudinal direction of the tube,
a portion of the tube has a second length between a proximal end of the first closing member and a distal end of the second closing member, and
the first length is longer than the second length.

19. The treatment system according to claim 8, wherein the first closing member and the second closing member are balloons.

20. The treatment system according to claim 8, further comprising a suction pump configured to suction fluid from the suction lumen.
